(19) United States
(12) Patent Application Publication
(10) Pub. No.: US 2018/0327447 A1
(43) Pub. Date: Nov. 15, 2018

(54) IMPROVED PROTEIN SEPARATION IN ION EXCHANGE CHROMATOGRAPHY

(71) Applicant: Merck Patent GmbH, Darmstadt (DE)

(72) Inventor: Matthias JOEHNCK, Muehltal (DE)

(73) Assignee: Merck Patent GmbH, Darmstadt (DE)

(21) Appl. No.: 15/776,941

(22) PCT Filed: Oct. 28, 2016
(86) PCT No.: PCT/EP2016/001803

§ 371 Date: May 17, 2018

(30) Foreign Application Priority Data
Nov. 18, 2015 (EP) 15195242.1

(51) Int. Cl. C07K 1/18; C07K 1/16; C07K 16/06; B01D 15/36

(52) U.S. Cl. CPC C07K 1/18; C07K 1/165; C07K 16/065; C07K 2317/52; B01D 15/363; C07K 2317/55; B01D 15/362

(57) ABSTRACT

Disclosed are improved preparative (>5 g/l) protein separations, which are achieved by combining salt and pH gradients for preparative protein separations in combination with the development of a preparative step elution protein separation based on data generated by combined salt-pH gradient runs.

The present invention relates to improved preparative (>5 g/l) protein separations. These improvements are achieved by combining salt and pH gradients for preparative protein separations in combination with the development of a preparative step elution protein separation based on data generated by combined salt-pH gradient runs.

### BACKGROUND

Protein heterogeneity is produced as a result of post translational modification in-vivo, or it is artificially induced via chemical and enzymatic reactions, or as a by-product in fermentation and purification processes due to mechanical stress, high temperature, or extreme pH [1-4]. Protein heterogeneity which is associated with mAb includes, but is not limited to, charge variants like acidic and basic variants, glycosylation variants, and size variants like aggregates, monomers, fragments, Fab, and Fc residues [5-7]. In therapeutic mAb, such product variants lead to diverse pharmacokinetics and pharmacodynamics, which will affect the stability, efficacy, and potency of the drug [1]. Therefore, they have to be thoroughly profiled and removed from the final product pool.

Liquid chromatography (LC) is used as the standard purification tool for mAb production [8]. Generic downstream process (DSP) for mAb includes, but is not limited to, protein A affinity chromatography (AC), ion exchange chromatography, and hydrophobic interaction chromatography (HIC) [9]. IEC (ion exchange chromatography) such as strong cation exchange chromatography (SCX), weak cation exchange chromatography (WCX), and weak anion exchange chromatography (WAX) are widely used at analytical scale to separate mAb charge variants with very similar isoelectric points (pl) and other protein variants, which include, but are not limited to, size variants, glycosylation variants, silylation variants, and C-terminal/N-terminal processed variants [7, 10-14]. While a shallow salt gradient slope using sodium chloride with fixed pH value can be used to characterize mAb variants, its application in charge variants separation is protein specific and has to be optimized for individual mAbs [15]. Chromatofocusing (CF) is the alternative to salt gradient in which a pH gradient is generated either internally of the column using polyampholyte buffers [16-21] or externally by mixing two appropriate buffers with different pH values at the column inlet, which subsequently travels through the column [22-26]. Depending on the respective pl values, mAb charge variants are focused at different points in the pH gradient hence resulting in highly resolved peaks [27].

Initial application of high-performance CF in IEC for mAb charge variants separation was limited to neutral and basic mAbs with pl range from 7.3 to 9.0 [28-29]. Recently, it was discovered that this application spectrum can be expanded to acidic mAbs (pl=6.2) by modulating the ionic strength in the pH gradient [29]. It is reported [29] that pH gradient at elevated and controlled ionic strength has led to better resolved peaks for the acidic, neutral, and basic mAb variants. While the above example depicts the success of salt mediated pH gradient for mAb charge variants separation at analytical scale, Kaltenbrunner et al. [30] have claimed much earlier that their pH-salt hybrid gradient using mannitol, borate, and sodium chloride is capable of separating mAb isoforms on preparative scale. They have used an ascending pH gradient from pH 7.0 to 9.1 combined with a descending salt gradient to separate isoproteins with pl between 8.15 and 8.70.

However, several limitations, drawbacks, and discrepancies are found in their approach. For example, the method suggested by them is only suitable for the separation of glycoprotein isoforms, which differed in several carbohydrate side-chains [29-30]. This confines the use of such gradient system only to glycated proteins thus making it unrealistic for other type of mAb variants like charge or size isoforms. Although it is claimed that the increased resolutions between the peaks are attributed to the pH-salt hybrid gradient, it remains unclear whether the unspecific reaction between the cis-diol containing oligosaccharides and the buffer component borate also has a significant effect on the improved separation [29]. Furthermore, their so-called “preparative” separation of the isoproteins was only 0.5 mg mAb per mL packed resin [30], which is still very low to be used in process-scale separation.

Up to date, process scale (≥30 mg/mL) mAb charge variants separation using pH-salt hybrid gradient system is reported for WCX—Fractogel® COO⁻ (M) [31]. However, the ascending pH gradient accompanied with an ascending salt gradient system is generated using acetate salt and it encompassed only a very narrow pH range of 5 to 6 thereby limiting this method towards mAb with an elution pH around 5.6. It should be noted that the pH range used in their pH-salt hybrid gradient is very close to the pKa of the carboxyl functional group (pKa=4.5). For WCX, it is known, that besides the buffering species used in the mobile phase, the functional groups on the resin backbone will also result in transient pH changes especially at pH near the pKa of the carboxyl group [32-33]. Since the pH range used in their study is very close to the pKa of the carboxyl group, it is reasonable to anticipate, that besides the acetate salt used in the mobile phase, the partially protonated carboxyl groups on the resin backbone will also exert certain buffering capacity to the gradient system. Furthermore, it is unclear whether the pH gradient in the hybrid pH-salt system is generated by the acetate buffer alone or whether it is a combined effect of carboxyl groups and acetate. Likewise, it is also uncertain whether this effect plays a major role in the charge variants separation. Also, the normal working pH range recommended for this type of resin is from 6 to 8 in which the carboxyl groups will be fully deprotonated (i.e. ionized). If it is worked at a pH value below 6, it is possible that the WCX will suffer a loss of capacity. Although high binding capacity between 38 and 54 g per L packed resin is reported in their study [31], this result is likely to be protein specific, which is coherent to their final message in the paper that the separation efficiency shown in their study is only specific to that particular antibody. The fact that no further separation example is given for pH above 6 and no other antibody has been used in their study makes the applicability of this method for the separation of other mAbs questionable.

Several patents [34-36] claim the use of CEX and mixed-mode chromatography (MMC) for mAb variant separation, which includes, but is not limited to, clearance of acidic, basic, deamidated, or glycol-variants from the mAb. Nevertheless, in these claims [34-36] mono gradient elution and step elution with a change of the salt concentration or of the pH value, once at a time, were applied. Furthermore, the feed comprised only one type of charge variant—acidic variant besides the product—mAb [34-36], which is relatively “pure”.

### Problem to be Solved

It is therefore an object of the present invention to provide an improved method for separation and purification of said proteins by use of ion exchange chromatography, which eliminates the described problems and disadvantages, and in particular, which takes into account that proteins include peptides, and especially that proteins include mAb, any mAb or other protein isoforms, charge variants, mAb fragments, mAb adducts, bispecific mAbs, any proteins derived partially from antibody constructs, such as Fabs, combination of mAbs with other proteins or smaller molecules, such as ADC's. This means, it is also an object of the present invention to separate these proteinacious products in order to separate the desired product in the highest possible purity.

In particular, it is an object of the present invention to provide a preparative method by which greater amounts of protein can be bound in a single pass to the chromatographic carrier material, and on the other hand, by which these proteins can be separated into the individual components and can be cleaned from unwanted ingredients.

### SUMMARY OF THE INVENTION

The present invention is thus directed to a method for purifying a protein from a mixture of proteins, by
a) providing a sample comprising at least two different proteins
b) applying this mixture to a ion exchange material with a total protein load ≥5 mg/ml, especially ≥30 mg/ml, in particular ≥60 mg/ml,
c) running an opposite pH-salt gradient by an ascending pH and descending salt concentration to separate proteins, or vice versa running a descending pH and an ascending salt concentration, or running a increasing pH gradient, or running a decreasing pH gradient,
d) using the separation data from c) to define and run a step elution profile for protein separation
   and
e) separating the proteins in a stepwise elution.

According to the invention, the separation of proteins can also be carried out in step d) in a gradient elution.

Thus, according to the invention, the mixture of proteins is therefore adsorbed or bound to an ion exchange material and eluted again.

Depending on the properties of the mixture of proteins to be separated the method for purifying may be performed using cation exchange materials, anion exchange materials or mixed mode chromatography materials.

The separation method of the present invention may be processed by inducing a pH gradient by applying a buffer system of at least two buffer solutions, whereby the needed adsorption or binding of proteins takes place in presence of one buffer solution and elution takes place in presence of increasing concentrations of the other buffer solution, while pH is ascending and the salt concentration is descending simultaneously or the other way round where the pH is descending and the salt concentration is ascending simultaneously. Suitable buffering systems for inducing a pH gradient use MES, MOPS, CHAPS, etc. and a conductivity alteration system using sodium chloride. In a modified form of the invention the applying of these buffer solutions inducing a pH gradient can be combined with an otherwise unchanged system or a system with a constant or gradually varying salt concentration.

Good separation results are found if in c) the pH is changed in the range from 4-10.5, and the salt concentration in the range of 0-1M salt.

The separation results are especially good if a pH gradient is induced by applying a buffer system adjusted between pH 5 and 9.5 and if a salt gradient is induced in a concentration range between 0-0.25 M.

The method according to the present invention as described before, is characterized by a pH gradient, which is induced by applying a buffer system of at least two buffer solutions and by adsorption or binding of proteins in presence of a first buffer solution and by elution in presence of increasing concentrations of another buffer solution, while the pH value is descending and the salt concentration is ascending simultaneously.

Particularly monoclonal antibodies (mAB) are separated from protein mixtures in a method according to the present invention. They are separated and purified from its associated charge variants, glycosylation variants, and/or soluble size variants, dimers and aggregates, monomers, ⅔ fragments, ¾ fragments, fragments in general, antigen binding fragments (Fab) and Fc fragments.

In summary, the present invention refers to a process wherein proteins, like monoclonal antibodies, are separated by use of opposite pH-salt gradients in ion exchange chromatography and utilising purification schemes, such as step elution purification in ion exchange chromatography. The purification schemes are developed utilizing opposite pH-salt gradients for identifying best operating conditions. As a result, an improved protein separation efficiency is made possible and a stepwise elution of desired proteins is possible at optimized conditions.

### DETAILED DESCRIPTION OF THE INVENTION

The invention disclosed here relates to opposite pH-salt hybrid gradient elution in ion exchange chromatography (IEC). More particularly, the invention is directed to the application of an ascending pH gradient in combination with a descending salt gradient for preparative separation of monoclonal antibodies (mAbs) from its associated charge variants (e.g. acidic and basic monomers), glycosylation variants, and/or soluble size variants (e.g. aggregates, monomers, ⅔-fragments, antigen-binding fragments (Fab), and crystallizable fragments (Fc)).

Unlike the mono gradient elution and step elution using salt or pH variations that are claimed in the patents described earlier [34-36], according to the present invention, an opposite pH-salt hybrid gradient comprised of an ascending pH gradient combined with a descending salt gradient is used in IEC, preferably CEX, and most preferred SCX for the separation of mAb variants like charge variants, glycosylation variants, ⅔ fragments, Fab, Fc, and aggregates from the product.

In contrary to the use of a relatively “pure” feed (only one charge variant type) as disclosed in these patents [34-36], the feeds of the present invention may comprise more than one charge variant types.

Thus, the biological solution comprising the protein substances, which shall be separated, is first mixed with an appropriate buffer solution. Then the received mixture is supplied to the ion exchange chromatography column and the charged groups and proteins, peptides or fragments, aggregates, isoforms and variants thereof are tightly bound to the strong cation exchange (SCX) stationary phase. To recover the analyte, the resin is then washed with a solvent neutralizing this ionic interaction. The neutralizing washing and elution is carried out with a mixture of suitable buffer solutions. Most preferred suitable biological buffers are selected from those providing a pH in the range between 4.5 and 10.5. Suitable buffers are already mentioned above. A number of suitable buffers can also be found on the internet under: http://www.hsbt.com.tw/pdf/Biological%20Buffers.pdf. Suitable buffers include preferably buffers known as MES, MOPS, CHAPS, HEPES. But there are also further buffers or buffer solutions that can be used, provided that they show no interfering reactions or interactions with the desired separation products or with separating materials.

A pH gradient separation at high loadings is possible because a low starting pH value allows a high protein binding capacity, especially on strong cation exchange resins. MAbs can be highly heterogeneous due to modifications such as sialylation, deamidation and C-terminal lysine truncation etc. Salt gradient cation exchange chromatography has been used with some success in characterizing mAb charge variants. However, additional effort is often required to tailor the salt gradient method for an individual mAb. In the fast-paced drug development environment, a more generic platform method is desired to accommodate the majority of the mAb analyses.

In 2009, Farnan and Moreno reported a method to separate mAb charge variants using pH gradient ion-exchange chromatography. The buffer employed to generate the pH gradient consisted of piperazine, imidazole, and Tris, covering a pH range of 5 to 9.5. While good separation was observed, the slope of the pH increase was shallow at the beginning and steep towards the end [15].

Now, through own experiments it was found, that an improved purification of protein A, mAbs and corresponding isoforms is possible in a novel pH gradient method combined with a salt gradient method for cation exchange chromatography. Several buffer species were selected for buffer formulation and the pH of the buffer was adjusted with sodium hydroxide. This method features a multi-component buffer system in which the linear gradient is run from 100% eluent of a low pH buffer (pH of about 5) to 100% eluent of a high pH buffer (pH of about 9.5 to 10.5). The concentration of each buffer species is adjusted to achieve a linear ascending or decending pH elution profile. Suitable buffer compositions are disclosed in the following examples. In addition to this, the provided examples also show how to combine the linear ascending pH gradient method with a descending linear salt gradient method for better separation using strong cation exchange resins. In order to confirm that a linear pH gradient is achieved a simple online pH meter can be used. The different buffer solutions can be provided in different containers and fed it into the column, so that the desired pH is set in the column. But it is also possible to mix appropriate quantities of the different buffer solutions from the containers together and to introduce the mixed buffer solution at an ascending pH during the course of separation into the column. This premixing of buffer solutions has the advantage that the pH value must not be adjusted in the separation column, and that a protein mixture bound to the ion exchanger is subjected to a uniformly changing pH.

Once the approximate pH elution range of the target mAb has been established in the initial run, further optimization of separation can simply be achieved by running a shallower pH gradient in a narrower pH range. Since strong cation exchange chromatography (SCX) is used, there is no interference of buffering effects from the stationary phase. The strong cation exchange (SCX) stationary phase usually is composed of a particulate or monolithic material, which contains groups that are negatively charged in aqueous solution. The interaction between these charged groups and proteins, peptides or fragments, aggregates or isoforms and variants thereof results in tightly binding of these basic analytes. In general said SCX materials possess sulfopropyl, sulfoisobutyl, sulfoethyl or sulfomethyl groups. Examples for such stationary phases are exchanger materials like Eshmuno® CPS, Eshmuno® CPX, or SP Fast Flow Sepharose®, Eshmuno® S Resin, Fractogel® SO₃(M), Fractogel SE Hicap (M), SP Cellthru BigBead Plus®, Streamline® SP, Streamline® SP XL, SP Sepharose Big Beads, Toyopearl® M-Cap II SP-550EC, SP Sephadex® A-25, Express-Ion® S, Toyopearl® SP-550C, Toyopearl® SP-650C, Source® 30S, Poros® 50 HS, Poros® 50 XS,

SP Sepharose Fast Flow, SP Sepharose® XL, Capto® S, Capto® SP ImRes, Capto® S ImpAct, Nuvia® HR-S, Cellufine® MAX S-r, Cellufine® MAX S-h, Nuvia® S, UNOsphere® S, UNOsphere® Rapid S, Toyopearl® Giga-Cap S-650 (M), S HyperCel Sorbent®, Toyopearl® SP-650M, Macro-Prep® High S, Macro-Prep® CM, S Ceramic HyperD® F, MacroCap® SP, Capto® SP ImpRes, Toyopearl® SP-650S, SP Sepharose® High Perform, Capto® MMC, Capto® MMC Imp Res, Eshmuno® HCX, Nuvia® High c-Prime or others.

SCX materials suitable for the separation process according to the present invention are particulate materials having mean particle diameters of >30 μm, preferably ≥40 μm, especially preferred in the range of 50-100 μm.

A suitable cation exchange (SCX) stationary phase and the buffer systems should be chosen in dependence of the pl of the protein. This means, that for eluting proteins bound to the ion exchange resin via non-covalent ionic interaction the ionic interaction must be weakened either by interaction with competing salts or by neutralization.

Alternatively and depending from operating conditions and pl of the proteins, also weak cation exchange resins, such as Fractogel® EMD COO (M), CM Sepharose HP, CM Sepharose® FF, Toyopearl® AF Carboxy 650-M, Macro-Prep® CM, Toyopearl® GigaCap CM, CM Ceramic Hyper® D, or Bio-Rex® 70 might be used.

Depending from the pl of the protein, anion exchange resins (SAX) might be used. Examples for strong anion exchange resins are Fractogel® EMD TMAE (M), Fractogel® EMD TMAE Medcap (M), Fractogel® EMD TMAE Hicap (M), Eshmuno® Q, Eshmuno® QPX, Eshmuno® QPX Hicap, Capto Q, Capto Q ImpRes, Q Sepharose® FF, Q Sepharose® HP, Q Sepharose® XL, Source® 30Q, Capto® Adhere, Capto® Adhere ImpRes, POROS® 50 HQ, POROS® 50 XQ, POROS® 50 PI, Q HyperCel, Toyopearl® GigaCap Q 650-M, Toyopearl® GigaCap Q 650-S, Toyopearl® Super Q, YMC® BioPro Q, Macro-Prep® High Q, Nuvia® Q or UNOsphere® Q.

Alternatively and depending from operating conditions and pl of the proteins also weak anion exchange resins carrying diethylaminoethyl (DEAE) of dimethylaminoethyl (DMAE) functionalities might be used. Examples are Fractogel® EMD DEAE, Fractogel® EMD DMAE, Capto® DEAE or DEAE Ceramic HyperD® F.

Now, as already mentioned above, unexpectedly it was found, that the separation of the comprising mixture of proteins, peptides or fragments, aggregates, isoforms and variants from the biological fluid can be carried out with excellent results by running an opposite pH-salt hybrid gradient, this means by an ascending pH and simultaneously descending salt concentration, or vice versa, to separate proteins. The gradient elution refers to a smooth transition of the salt concentration in the elution buffer with changing pH, here mainly from a high to low salt concentration. In order to generate appropriate conditions for this separation process both buffer solutions are mixed with suitable salt concentrations.

These conditions of an opposite pH-salt hybrid gradient allows to separate multiple consecutive fractions in an improved resolution and collecting them while elution conditions, pH and salt concentration, are adjusted in a linear fashion. An opposite pH-salt linear gradient offers the highest resolution for ion exchange chromatography and hydrophobic interaction chromatography and a large number of consecutive fractions may be collected.

To carry out the separation according to the present invention a high salt concentration is preferably added to the buffer solution having a low pH. The buffer solution with a high pH is preferably used without the addition of salt. If the resulting two buffer solutions are mixed together gradually and are introduced gradually directly after mixing into the separating column the pH of the elution solution increases over time while the salt concentration decreases at the same time.

In general, NaCl is a useful salt for conducting the binding and elution process of the different protein fractions because the changing NaCl concentration is combined with a changing conductivity, which influences the binding strength of charged groups of proteins bound to the ion exchanger.

Once appropriate conditions of an opposite pH-salt hybrid gradient for the separation a proteinaceous mixture is established the individual peaks of the different protein fractions can be assigned to optimal conditions under which separation takes place from the mixture. These conditions can be used for stepwise elution of each desired protein fraction. In the following examples, the application of this principle is shown.

Below, experiments are exemplified wherein separations of at least three product charge variants and at least three product size variants are performed. It is found that these variants as listed before are successfully resolved according to the present invention in a single chromatographic run.

This surprising separation result can be achieved if a simple buffer system is used instead of polyampholyte buffer to cover a wide pH range from 4.5 to 10.5 and if sodium chloride is used to induce the salt gradient. The opposite pH-salt hybrid gradient is generated by externally mixing two buffers (i.e. A and B) with different pH values and sodium chloride concentrations (i.e. A with low pH and high salt concentration; B with high pH and low salt concentration) at the column inlet, which then travels through the column.

The experiments have shown that both at low load and at very high loading a good separation can be achieved with various proteins when the process is controlled accordingly. The results achieved at very high loading are particularly convincing because in general there is an early breakthrough and a proper separation of proteins is not possible.

Exemplary multiproduct separation examples are given for three different feeds containing various mAb isoproteins at low loading (≈1 mg/mL packed resin), at higher loading (≥30 mg/mL), and at very high loading (≥60 mg/mL). For the separation different gradient types were tested like salt gradient, pH gradient, parallel pH-salt hybrid gradient, and opposite pH-salt hybrid gradient. Results at low loading showed that the salt gradient is suitable for separation of size variants separation (i.e. for aggregate and monomer), whereas a pH gradient is suitable for charge variants separation (i.e. for acidic, neutral, and basic monomers). Surprisingly the best separation for both, size and charge variants, is achieved in the opposite pH-salt hybrid gradient system.

Another surprising result of these experiments was that the higher loading with a protein load ≥30 mg/mL allowed good separation in preparative scale without suffering in a loss of separation efficiency.

The results of numerous experiments suggest the use of an ascending pH gradient with a descending salt gradient so that the protein variants will experience not only the focusing effects in the linear pH gradient but concomitantly also the retardation in the protein migration velocities due to decreasing salt concentration thereby resulting in an improved resolution. Also Zhou et al. [31] have utilized sodium acetate as the only buffering component and at the same time they have used the same salt at elevated concentration to generate an ascending conductivity gradient. Thus, they have used only one salt type to concomitantly generate a parallel increasing pH and conductivity gradient. Due to the pKa of acetate, the pH gradient which they generated using this type of buffering system is only limited to a pH range between 4.8 and 6.2 [29, 31]. Contrary to this, the present experiments show, that advantageous results are achieved if the mobile phase is composed of a buffering system using MES, MOPS, CHAPS, etc. and a conductivity alteration system using sodium chloride. Thus, the core of the present invention is not comparable with what is suggested by Zhou et al. [31]. The hybrid gradient system of the present invention utilizes common buffer systems, which cover a wide pH range from 4.5 to 10.5. This provides an advantage for the separation of a broad range of mAbs with acidic, neutral, or basic pl values. Since SCX is used, there is no interference of buffering effects from the stationary phase compared to the WCX with carboxyl ligands in the pH range from 4.5 to 10.5. In comparison to the pH-salt hybrid system described by Kaltenbrunner et al. [30], whose buffer system utilizes hydroxyl ions, which are liberated in the reaction of cis-diol groups of mannitol with borate achieving an acidic pH value in the mobile phase, the system of the present invention applying a simple buffer system is fundamentally different. A particular advantage of the present invention is that there is no unspecific binding between the buffer components in the mobile phase and the proteins like in the case with the borate buffer. In DSP a high dynamic binding capacity is always preferred. Meanwhile, product pool with low conductivity is also desirable, so that the eluent can be loaded directly onto the next IEC if required, which can save the need for an intermediate dilution or desalting step. The opposite hybrid pH-salt gradient system, which is disclosed here, serves these purposes very well, because it has been found, that the dynamic binding capacity (DBC) increases, if some salts are added into the starting buffer solution and elution at lower conductivity becomes possible with the descending salt gradient. Yet a good separation between the protein variants is facilitated via the chromatofocusing effects of the ascending pH gradient. And last but not least, it has to be mentioned, that the method disclosed here is suitable for mAb variants separation in preparative scale with protein load 30 mg/mL without suffering in a loss of separation efficiency. In addition to this, the separation process using gradient elution can be directly transferred into step elution using similar buffer systems. Furthermore, the high protein load further strengthens the usefulness of the present invention.

A variety of experiments have been carried out from which a selection of examples is disclosed below. These examples show how varied the claimed method may be carried out. Through simple adjustments of the process parameters, it is possible to separate and purify different protein fractions, whose separation is in general difficult. Thus, it is possible to change the pH gradient less or to change the salt concentration by only a few millimoles. Another variant consists in choosing the chromatography material. In general, cation exchange materials are suitable, like Eshmuno® CPX, but depending on the desired separation it is also possible to use anion exchange materials or mixed mode chromatography materials (MMCs). Mixed-mode chromatography materials contain ligands of multimodal functionality that allow protein adsorption by a combination of ionic interactions, hydrogen bonds, and/or hydrophobic interactions. A suitable mixed mode separation material is Eshmuno® HCX. Hence. also the use of different ion exchange materials result in characteristic separations of different protein fractions.

Suitable anion exchange materials for protein separation and purification are commercially available, for example Sepharose Q™ FF (Amersham-Biosciences/Pharmacia), Capto® Q ImpRes, DEAE Sepharose® Fast Flow, Q Sepharose Fast Flow, (GE-Healthcare), Fractogel® EMD DEAE(M), Fractogel® EMD TMAE(M), Eshmuno® Q (Merck KGaA), Econo-Pac® (Bio-Rad), Ceramic HyperD or others. Depending on the protein mixture

Depending on the protein mixture and on the comprising impurities, another ion exchanger may lead to the best separation results.

The present description enables the person skilled in the art to apply the invention comprehensively. Even without further comments, it is assumed that a person skilled in the art will be able to utilise the above description in the broadest scope.

Practitioners will be able, with routine laboratory work, using the teachings herein, to separate proteins as defined above efficiently in the new process utilising purification schemes, such as step elution purification in ion exchange chromatography, developed utilizing opposite pH-salt gradients for identifying best operating conditions.

If anything is still unclear, it is understood that the publications and patent literature cited should be consulted. Accordingly, these documents are regarded as part of the disclosure content of the present description.

For better understanding and in order to illustrate the invention, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying to the person skilled in the art that, both in the examples given and also in the remainder of the description, the component amounts present in the compositions always only add up to 100% by weight or mol-%, based on the composition as a whole, and cannot exceed this, even if higher values could arise from the percent ranges indicated. Unless indicated otherwise, % data are % by weight or mol-%, with the exception of ratios, which are shown in volume data, such as, for example, eluents, for the preparation of which solvents in certain volume ratios are used in a mixture.

The temperatures given in the examples and the description as well as in the claims are always in ° C.

### REFERENCES

1. A. J. Chirino; A. Mire-Sluis; Characterizing biological products and assessing comparability following manufacturing changes; Nat.

Biotechnol. 22 (2004) 1383-1391.
2. N. Jenkins; Modifications of therapeutic proteins: challenges and prospects; Cytotechnology 53 (2007) 121-125.
3. J. Vlasak; R. Ionescu; Fragmentation of monoclonal antibodies, MAbs 3 (2011) 253-263.
4. M. Haberger; et al. Assessment of chemical modifications of sites in the CDRs of recombinant antibodies. Susceptibility vs. functionality of critical quality attributes, MAbs 6 (2014) 327-339.
5. R. J. Harris; Processing of C-terminal lysine and arginine residues of proteins isolated form mammalian cell culture; J. Chromatogr. A 705 (1995) 129-134.
6. W. Wang; Protein aggregation and its inhibition in biopharmaceutics, Int. J. Pharm. 289 (2008) 1-30.
7. S. Chen, H. Lau, Y. Brodsky, G. R. Kleemann, R. F. Latypov, The use of native cation-exchange chromatography to study aggregation and phase separation of monoclonal antibodies, Protein Sci. 19 (2010) 1191-1204.
8. P. Gronemeyer; R. Ditz; J. Strube; Trends in upstream and downstream process development for antibody manufacturing; Bioengineering 1 (2014) 188-212.
9. R. L. Fahrner; H. L. Knudsen; C. D. Basey; W. Galan; D. Feuerhelm; M. Vanderlaan; G. S. Blank; Industrial purification of pharmaceutical antibodies: Development, operation, and validation of chromatography processes; Biotechnol. Genet. Eng. Rev. 18 (2001) 301-327.
10. X. Kang, D. D. Frey, High-performance cation-exchange chromatofocusing of proteins, J. Chromatogr. A 991 (2003) 117-128.
11. T. M. Pabst, G. Carta, N. Ramasubramanyan, A. K. Hunter, P. Mensah, M. E. Gustafson, Separation of protein charge variants with induced pH gradients using anion exchange chromatographic columns, Biotechnol. Prog. 24 (2008) 1096-1106.
12. L. I. Tsonev, & A. Hirsh, Theory and applications of a novel ion exchange chromatographic technology using controlled pH gradients for separating proteins on anionic and cationic stationary phases, J. Chromatogr. A 1200 (2008) 166-182.
13. L. I. Tsonev, & A. Hirsh, Improved resolution in the separation of monoclonal antibody isoforms using controlled pH gradients in IEX chromatography, Am. biotechnol. Lab. 27 (2009) 10-12.
14. H. Lau, et al., Investigation of degradation processes in IgG1 monoclonal antibodies by limited proteolysis coupled with weak cation-exchange HPLC, J. Chromatogr. B 878 (2010) 868-876.
15. D. Farnan & G. T. Moreno, Multiproduct High-resolution monoclonal antibody charge variant separations by pH gradient ion-exchange chromatography, Anal. Chem. 81 (2009) 8846-8857.
16. L. Sluyterman, & O. Elgersma, Chromatofocusing: Isoelectric focusing on ion-exchange columns I. General principles, J. Chromatogr. 150 (1978) 17-30.
17. L. Sluyterman & J. Wijdenes, Chromatofocusing: Isoelectric focusing on ion-exchange columns II. Experimental verification, J. Chromatogr. 150 (1978) 31-44.
18. L. Sluyterman & J. Wijdenes, Chromatofocusing: IV. Properties of an agarose polyethyleneimine ion exchanger and its suitability for protein separations columns, J. Chromatogr. A 206 (1981) 441-447.
19. L. Sluyterman, & C. Kooistra, Ten years of chromatofocusing: a discussion, J. Chromatogr. A 470 (1989) 317-326.
20. D. D. Frey, A. Barnes, J. Strong, Numerical studies of multicomponent chromatography employing pH gradients, AIChE J. 41 (1995) 1171-1183.
21. D. D. Frey, Local-equilibrium behavior of retained pH and ionic strength gradients in preparative chromatography, Biotechnol. Prog. 12 (1996) 65-72.
22. R. Mhatre, W. Nashabeh, D. Schmalzing, X. Yao, M. Fuchs, D. Whitney, F. Regnier, Purification of antibody Fab fragments by cation-exchange chromatography and pH gradient elution, J. Chromatogr. A 707 (1995) 225-231.
23. T. Andersen, M. Pepaj, R. Trones, E. Lundanes, T. Greibrokk, Isoelectric point separation of proteins by capillary pH-gradient ion-exchange chromatography, J. Chromatogr. A 1025 (2004) 217-226.
24. T. Ahamed, et al., Selection of pH-related parameters in ion-exchange chromatography using pH-gradient operations, J. Chromatogr. A 1194 (2008) 22-29.
25. Rozhkova, Quantitative analysis of monoclonal antibodies by cation-exchange chromatofocusing, J. Chromatogr. A 1216 (2009) 5989-5994.
26. X. Kang, J. P. Kutzko, M. L. Hayes, D. D. Frey, Monoclonal antibody heterogeneity analysis and deamidation monitoring with high-performance cation-exchange chromatofocusing using simple, two component buffer systems, J. Chromatogr. A 1283 (2013) 89-97.
27. J. C. Rea, G. T. Moreno, Y. Lou, D. Farnan, Validation of a pH gradient-based ion-exchange chromatography method for high-resolution monoclonal antibody charge variant separations, J. Pharm. Biomed. Anal. 54 (2011) 317-323.
28. D. Farnan, G. T. Moreno, Multiproduct high-resolution monoclonal antibody charge variants separations by pH gradient ion-exchange chromatography, Anal. Chem. 81 (2009) 8846-8857.
29. L. Zhang, T. Patapoff, D. Farnan, B. Zhang, Improving pH gradient cation-exchange chromatography of monoclonal antibodies by controlling ionic strength, J. Chromatogr. A 1272 (2013) 56-64.
30. Kaltenbrunner, C. Tauer, J. Brunner, A. Jungbauer, Isoprotein analysis by ion-exchange chromatography using a linear pH gradient combined with a salt gradient, J. Chromatogr. 639 (1993) 41-49.
31. J. X. Zhou, S. Dermawan, F. Solamo, G. Flynn, R. Stenson, T. Tressel, S. Guhan, pH-conductivity hybrid gradient cation-exchange chromatography for process-scale monoclonal antibody purification, J. Chromatogr. A 1175 (2007) 69-80.
32. T. M. Pabst, G. Carta, pH transitions in cation exchange chromatographic columns containing weak acid groups, J. Chromatogr. A 1142 (2007) 19-31.
33. T. M. Pabst, G. Carta, N. Ramasubramanyan, A. K. Hunter, Protein separations with induced pH gradients using cation-exchange chromatographic columns containing weak acid groups, J. Chromatogr. A 1181 (2008) 83-94.
34. C. D. Basey, G. S. Blank, Protein purification by ion exchange chromatography, International patent WO 99/57134 (1999).
35. J. Burg, B. Hilger, T. Kaiser, W. Kuhne, L. Stiens, C. Wallerius, F. Zetti, Optimizing the production of antibodies, International patent WO 2011/009623 A1 (2011).
36. N. Ramasubramanyan, L. Yang, M. O. Herigstad, H. Yang, Protein purification methods to reduce acidic species, U.S. patent US 2013/0338344 A1 (2013).

### EXAMPLES

### Example 1

Preparative Separation of mAb A Charge Variants Using IEC

The preparative chromatographic runs are performed as follows: Equipment: ÄKTApurifier 100

Column: Eshmuno® CPX, Merck Millipore, mean particle size 50 μm, ionic capacity 60 μmol/mL, column dimension 8 i.d.×50 mm (2.5 mL)

Feed: MAb A post protein A pool

Mobile phase:
(A) Buffers for linear salt gradient consisted of 10 mM MES. Buffer A without NaCl. Buffer B with 1 M NaCl. pH of both buffers were adjusted to pH 6.5 with NaOH.
(B) Buffers for linear pH gradient consisted of 12 mM acetic acid, 10 mM MES, 6 mM MOPS, 4 mM HEPES, 8 mM TAPS, 8 mM CHES, 11 mM CAPS, 53 mM NaOH. No NaCl is added into buffer A and B unless stated in the description of the figures. Buffer A is adjusted to pH 5 with HCl. No pH adjustment was needed for buffer B (pH=10.5).
(C) Buffers for opposite pH-salt hybrid gradient with descending pH and ascending salt gradient consisted of 12 mM acetic acid, 12 mM acetic acid, 10 mM MES, 6 mM MOPS, 4 mM HEPES. Buffer A without NaCl and pH was adjusted to 8 with NaOH. Buffer B with 200 mM NaCl and pH was adjusted to 5 with NaOH.
(D) Buffers for opposite pH-salt hybrid gradient with ascending pH and descending salt gradient consisted of 12 mM acetic acid, 12 mM acetic acid, 10 mM MES, 6 mM MOPS, 4 mM HEPES, 8 mM TAPS, 8 mM CHES, 11 mM CAPS. Buffer A with 150 mM NaCl and pH was adjusted to 5 with NaOH. Buffer B without NaCl and pH was adjusted to 10.5 with NaOH.
(E) Buffers for parallel pH-salt hybrid gradient with ascending pH and ascending salt gradient consisted of 12 mM acetic acid, 10 mM MES, 6 mM MOPS, 4 mM HEPES. Buffer A without NaCl and pH was adjusted to 5 with NaOH. Buffer B with 200 mM NaCl and pH was adjusted to 8 with NaOH.

Linear Gradient Elution:

Gradient Slope: 60 CV (2.5 mL/CV), otherwise will be stated in the description of the figures

Flow rate: 1 mL/min (=119 cm/h)

Protein load: 1 mg/mL, otherwise will be stated in the descriptions of the figures

Cleaning-In-Place (CIP): 0.5 M NaOH (3-5 CV)

Step Elution:

Flow rate: 1 mL/min (=119 cm/h) was used to bind protein; 3 mL/min (=358 cm/h) was used to elute protein

Protein load: 30 mg/mL

Cleaning-In-Place (CIP): 0.5 M NaOH (3-5 CV)

Buffer A and B as stated in (D) (see mobile phase) are used. Zero % buffer B is used for protein binding. For protein elution different steps are generated by mixing buffer A and B at different concentrations as follows:

| | Buffer |
|---|---|
| Step | B [%] |
| 1 | 46 |
| 2 | 55 |
| 3 | 70 |
| 4 | 81 |
| 5 | 89 |
| 6 | 93 |

Analytics are performed as follows:

Equipment: ÄKTAmicro

Size-exclusion high performance liquid chromatography (SE-HPLC) is performed using BioSep™-SEC-s3000, Phenomenex, column dimension 7.8 i.d.×300 mm, particle size 5 μm. Buffer used consists of 50 mM NaH₂PO₄ and 300 mM NaCl, pH 7. Isocratic elution at a flow rate of 1 mL/min is used. Injection volume varies from 40 μL to 100 μL.

Cation exchange high performance liquid chromatography (CEX-HPLC) is performed using YMC BioPro Sp-F, YMC Co. Ltd., column dimension 4.6 i.d.×50 mm, particle size 5 μm. Buffers as described previously in (B) are used. Gradient elution from 50% to 85% buffer B in 8.75 CV gradient lengths at a flow rate of 0.7 mL/min was used. Injection volume varies from 40 μL to 100 μL.

Results:

The following data is collected to compare the efficiencies of different gradient types in separating mAb A charge variants using CEX.

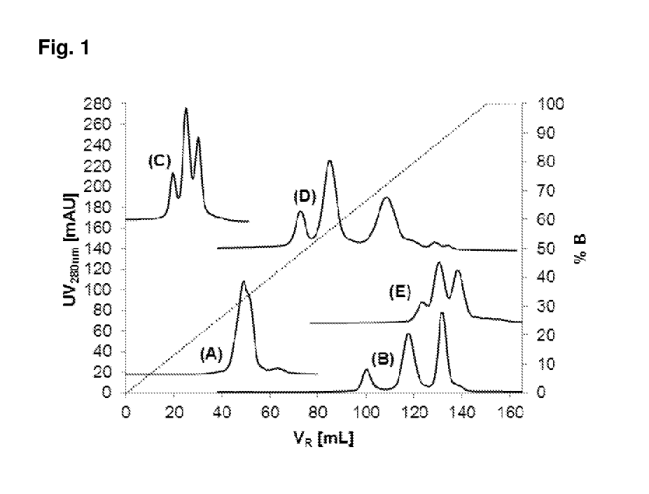

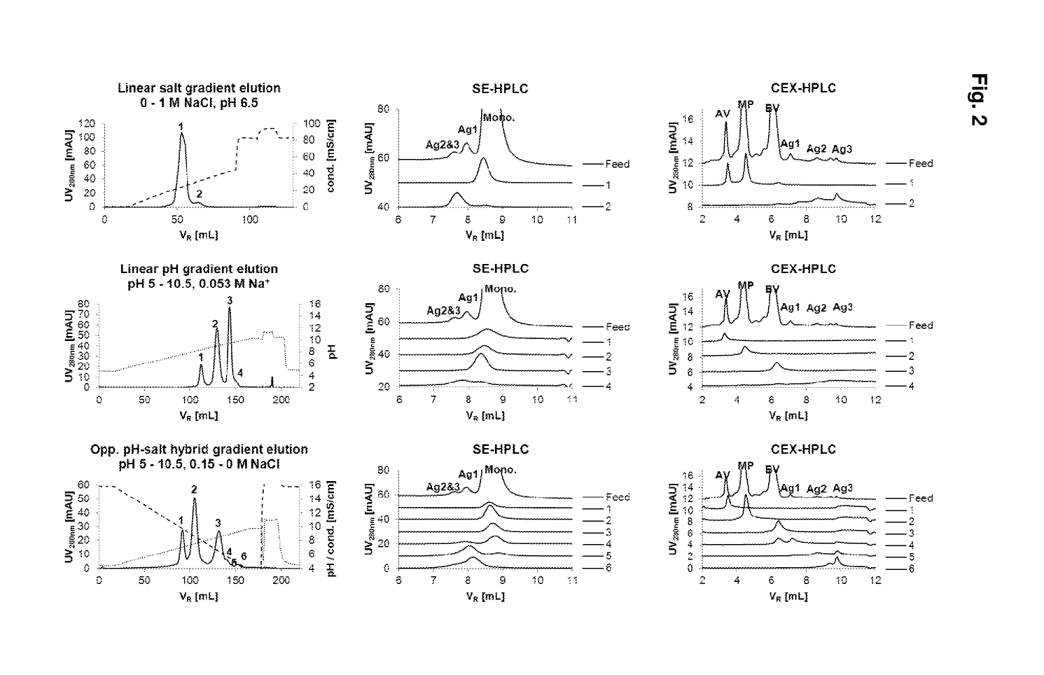

In FIG. 1 (FIG. 1) the screening of different gradient elution types for the separation of mAb A charge variants are shown. (A) Linear salt gradient elution: 0-1 M NaCl, pH 6.5, (B) Linear pH gradient elution: pH 5-10.5, 0.053 M Na⁺, (C) Opposite pH-salt hybrid gradient elution with descending pH and ascending salt gradient: pH 8-5, 0-1 M NaCl, (D) Opposite pH-salt hybrid gradient elution with ascending pH and descending salt gradient: pH 5-10.5, 0.15-0 M NaCl, (E) Parallel pH-salt hybrid gradient elution with ascending pH and ascending salt gradient: pH 5-8, 0-0.2 M NaCl on Eshmuno® CPX. Counter-ions originated from sodium hydroxide (used for pH adjustment of the buffer) are depicted as Na⁺ whereas those from sodium chloride are depicted as NaCl.

Among all the gradient elution runs depicted in FIG. 1, the opposite pH-salt hybrid gradient in (D) show the highest number of resolved peaks—6, while the other two hybrid gradients (C) and (E) showe moderately resolved peaks (number of peaks—3). Classical elution methods like the linear pH gradient (B) show three highly resolved peaks with a shoulder at the end whereas linear salt gradient only show two peaks.

The following data shows the detailed HPLC analyses of the fractions pooled in gradient type (A), (B) and (D) of FIG. 1.

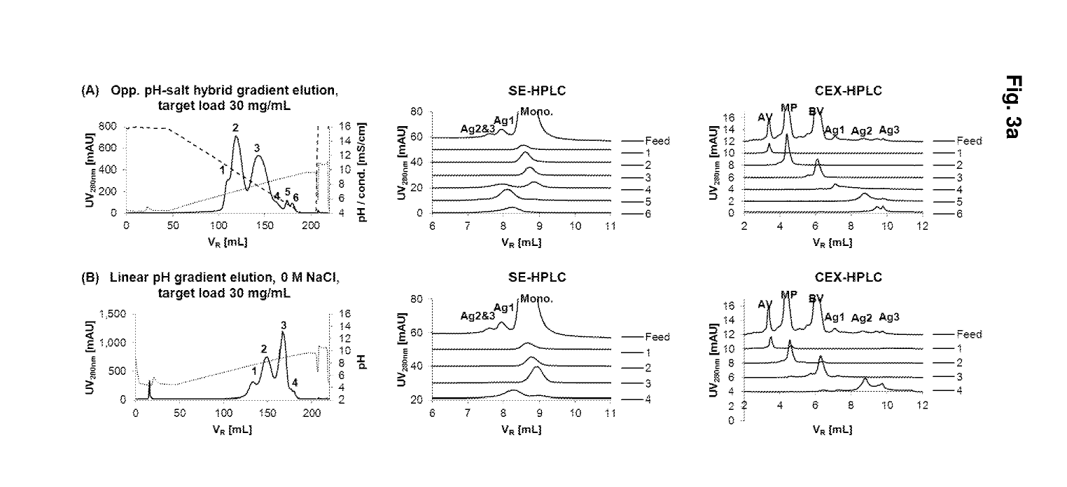

In FIG. 2 (FIG. 2) the left column depicts the respective preparative chromatographic runs shown and described in FIG. 1 (A), (B) and (D) from top to bottom (dashed line: conductivity (cond.), dotted line: pH). Middle and right columns are the HPLC analyses of the individual peaks pooled from the respective preparative chromatographic runs on the left. Mono.—monomer, Ag 1, 2, and 3—aggregate variants 1, 2, and 3, AV—acidic charge variant, MP—main peak, BV—basic charge variants. Counter-ions originated from sodium hydroxide (used for pH adjustment of the buffer) are depicted as Na⁺ whereas those from sodium chloride are depicted as NaCl.

For all three gradient elution types selected, it is observed that the aggregates can be resolved from the monomers (see SE-HPLC in FIG. 2). According to the preparative chromatograms in FIG. 2, linear salt gradient elution provides slightly better resolved aggregate peak (peak number 2) from monomer peak (peak number 1). However, there is absolutely no separation of the charge variants except the basic charge variants (see CEX-HPLC in FIG. 2). Linear pH gradient and opposite (Opp.) pH-salt hybrid gradient with ascending pH and descending salt gradient depict highly resolved acidic (AV) and basic charge variant (BV) peaks from the main peak (MV). In addition to charge variants separation, the opposite pH-salt hybrid gradient also depicts three separate aggregate peaks, which demonstrates the advantage of this type of hybrid gradient.

The following data compare the capacity as well as the corresponding isoproteins separation efficiencies of the opposite pH-salt hybrid gradient elution and linear pH gradient.

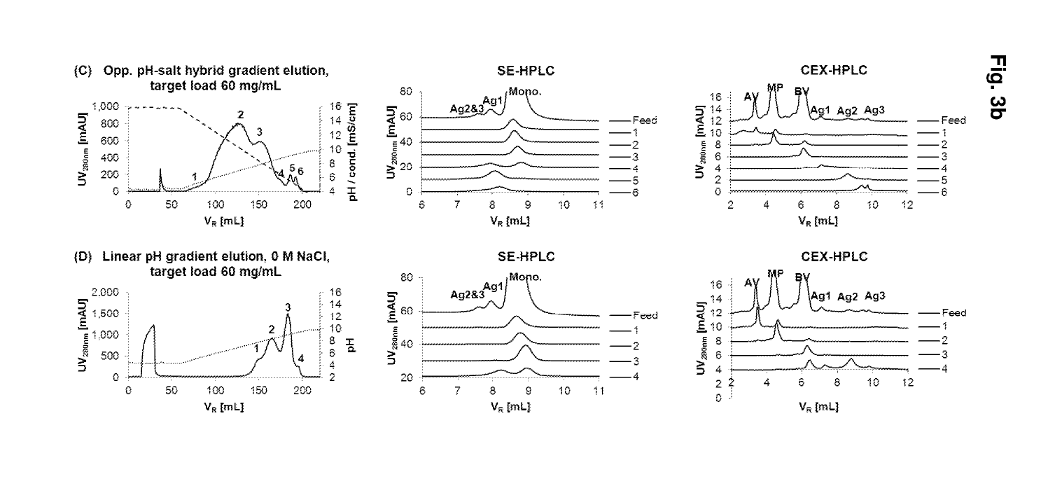

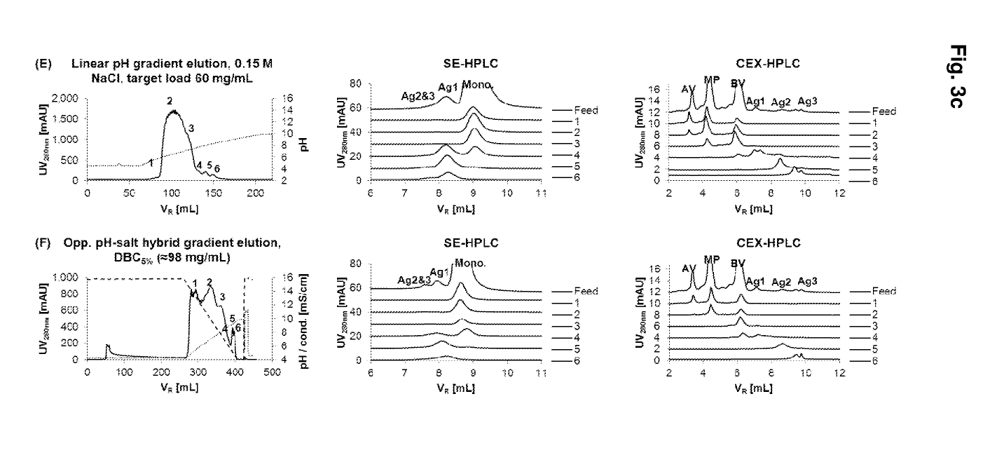

FIG. 3a-3d (FIG. 3a 3d): Left column depicts the respective preparative chromatographic runs of opposite pH-salt hybrid gradient pH 5-10.5, 0.15-0 M NaCl (A, C, F, G), linear pH gradient pH 5-10.5, 0.053 mM Na⁺ (B, D), and linear pH gradient with salt pH 5-10.5, 0.15 M NaCl (E) on Eshmuno® CPX, using different target loads. For (A)-(F) gradient slope was 60 CV whilst for (G) it is 276 CV. Dashed line—conductivity (cond.), dotted line-pH. Middle and right columns are the HPLC analyses of the individual peaks pooled from the respective preparative chromatographic runs on the left. Mono.—monomer, Ag 1, 2, and 3—aggregate variants 1, 2, and 3, AV—acidic charge variant, MP—main peak, BV—basic charge variants. Counter-ions originated from sodium hydroxide (used for pH adjustment of the buffer) are depicted as Na⁺ whereas those from sodium chloride are depicted as NaCl. Protein recovery for every run is >90%.

When a target load of 30 mg/mL packed resins is used, breakthrough of protein is observed for the linear pH gradient system (see (B) in FIG. 3) whereas this is not observed for the opposite pH-salt hybrid gradient system (see (A) in FIG. 3). When the target load is increased to 60 mg/mL packed resins, the breakthrough of protein increases to about 80% (100% UV signal for the feed≈1560 mAU) for the linear pH gradient system (see (D) in FIG. 3). It should be noted that at the same target load of 60 mg/mL packed resins, there is no breakthrough of protein observed in the opposite pH-salt hybrid gradient system. The peak between V_{R}˜40 and 50 mL (see (C) in FIG. 3) occurrs when the sample injection is finished. (i.e. when the column is washed with the binding buffer). To confirm that the dynamic binding capacity (DBC) can be increased with elevated salt concentration, the pH gradient elution experiment is repeated by adding 0.15 M sodium chloride into both buffer A and B and the result in (E) shows that the target load of 60 mg/mL packed resins is achieved without any protein flowing through the column. Nevertheless, in terms of separation efficiency, at 60 mg/mL load the fractions pooled in the opposite pH-salt hybrid gradient (see CEX-HPLC of (C) in FIG. 3) show higher purities of the individual variant species compared to that of the pH gradient with 0.15 M NaCl (see CEX-HPLC of (E) in FIG. 3). Also the main peak 2 and the basic charge variant peak 3 are better resolved in the opposite pH-salt hybrid gradient than in the pH gradient at elevated salt concentration (compare preparative chromatograms (C) and (E) in FIG. 3).

For the opposite pH-salt hybrid gradient system, the dynamic binding capacity at 5% breakthrough (DBC_{5%}) is found to be approximately 98 mg/mL packed resins (see (F) in FIG. 3). To investigate the separation efficiency between different gradient slopes, the same DBC_{5%} experiment was repeated using a very shallow gradient—276 CV (see (G) in FIG. 3). Besides the higher resolution between the individual peaks in the shallow gradient, no significant improvement in the purities of the respective pools is observed compared to the steeper slope (compare CEX-HPLC of (F) and (G) in FIG. 3. Besides the significant increase in the binding capacity, the opposite pH-salt hybrid gradient system also supports the high resolution separation of acidic and basic charge variants from the main peak. Compared to the classical pH gradient elution, the opposite pH-salt hybrid gradient system provides the following benefits: higher binding capacity (at least two to three fold), comparable if not better separation between product associated charge variants, and significant improved separation between product associated aggregate species.

It should be noted that the initial salt concentration in the opposite pH-salt of 150 mM is relatively high for preparative CEX resins. It is reasonable to anticipate that if lower salt concentration is used (e.g. 50 mM or 100 mM) higher binding capacity with improved resolutions between the peaks can be attained.

The following shows the transfer of separation process from hybrid pH-salt gradient elution into a series of stepwise elution using the same buffer systems.

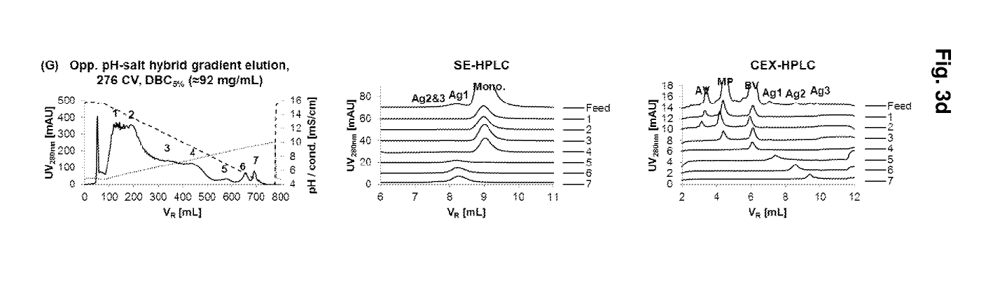

FIG. 4: (FIG. 4) Left column depicts the multiproduct separation using step elution on Eshmuno® CPX. Peak 1 and 2 are eluted in the first step (46% buffer B), peak 3 in the second step (55% buffer B), peak 4 in the third step (70% buffer B), peak 5 in the fourth step (81% buffer B), peak 6 in the fifth step (89% buffer B), and peak 7 in the sixth step (93% buffer B). Dashed line—conductivity (cond.), dotted line-pH. Middle and right columns are the HPLC analyses of the individual peaks pooled from the preparative chromatographic run on the left. Mono.—monomer, Ag 1, 2, and 3—aggregate variants 1, 2, and 3, AV—acidic charge variant, MP—main peak, BV—basic charge variants.

Based on the elution profile in (A) of FIG. 3, the respective concentrations of buffer B at which each variant species are eluted are transferred into a series of stepwise elution using the same buffer system. As seen in FIG. 4, the individual product variants are very well separated from each other via step elution. Beside the good separation, more than 80% yields (according to the areas under the peaks in CEX-HPLC of FIG. 4) of the respective monomeric species (i.e. AV, MP, and BV) are achieved in peak 1, 2, and 3.

The ease of transferring the separation process from gradient elution into step elution strengthens the advantage of the opposite pH-salt hybrid gradient for process development of multiproduct separation in the shortest time using the least empirical efforts.

### Example 2

Preparative Separation of mAb B Charge Variants Using IEC

The preparative chromatographic runs are performed as follows:

Equipment: ÄKTApurifier 100

Column: Eshmuno® CPX, Merck Millipore, mean particle size 50 μm, ionic capacity 60 μmol/mL, column dimension 8 i.d.×20 mm (1 mL)

Feed: MAb B monomer post protein A pool

Mobile phase:
(A) For linear salt gradient, buffer A and B consist of 20 mM acetic acid. In buffer B is added with 250 mM sodium chloride whereas none was added to buffer A. Both buffers were adjusted to pH 5 with NaOH.
(B) For linear pH gradient, buffer A consisted of 12 mM acetic acid, 10 mM MES, and 10 mM MOPS whilst buffer B consisted of 6 mM MOPS, 6 mM HEPES, 10 mM TAPS, and 9 mM CHES. Buffer A and B are adjusted to pH 5 and 9.5, respectively with NaOH.
(C) For opposite pH-salt hybrid gradient with ascending pH and descending salt gradient, same buffer components as (A) are used but a certain amount of sodium chloride (50 mM or 100 mM) is added into buffer A while none was added to buffer B. Both buffers were adjusted to pH 5 and 9.5, respectively with NaOH.

Gradient Slope: 60 CV (1 mL/CV), otherwise will be stated in the descriptions of the figures

Flow rate: 1 mL/min (=119 cm/h)

Protein load: 1 mg/mL, otherwise will be stated in the descriptions of the figures

CIP: 0.5 M NaOH (3-5 CV)

Analytics are performed as follows:

Equipment: ÄKTAmicro

CEX-HPLC is performed using YMC BioPro Sp-F, YMC Co. Ltd., column dimension 4.6 i.d.×50 mm, particle size 5 μm. Buffers comprised of 10 mM MES, 6 mM MOPS, 4 mM HEPES, 8 mM TAPS, 8 mM CHES, and 31.8 mM NaOH. Buffer A is adjusted to pH 6 with HCl. No pH adjustment is needed for buffer B (pH=9.5). Gradient elution from 25% to 60% buffer B in 15.76 CV gradient lengths at a flow rate of 0.7 mL/min is used. Injection volume varied from 40 μL to 100 μL.

Results:

The following data compare the isoproteins separation efficiencies of three different gradient elution systems: Linear salt gradient elution, linear pH gradient elution, and opposite pH-salt hybrid gradient elution on CEX.

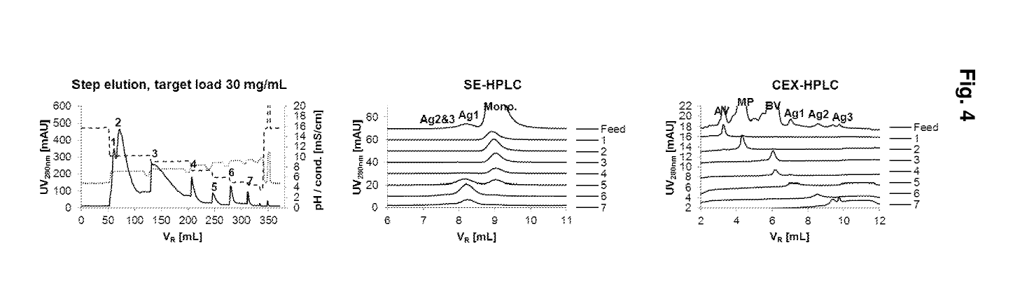

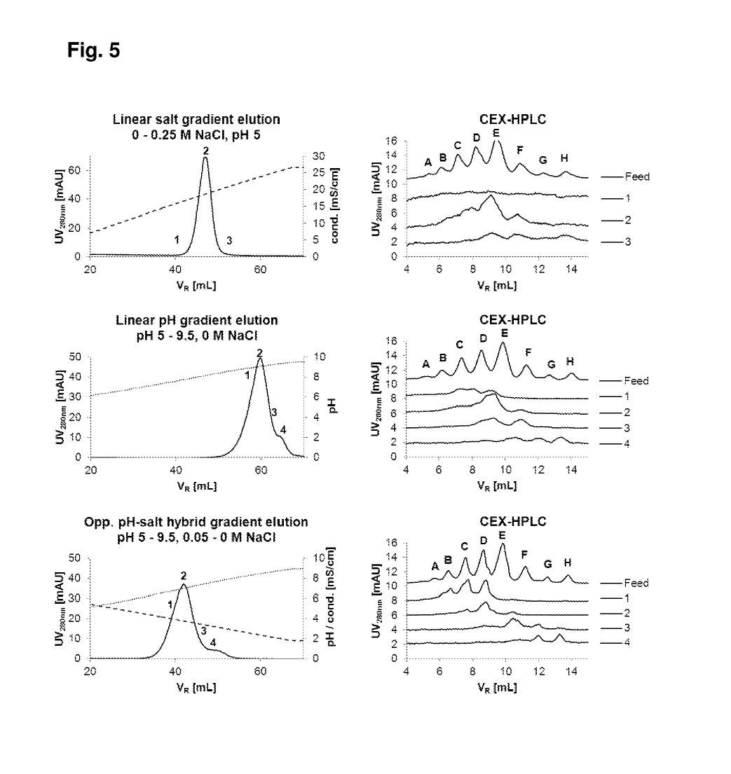

FIG. 5: (FIG. 5) Left column depicts the respective preparative chromatographic runs of three linear gradient elution types on Eshmuno® CPX. Dashed line—conductivity (cond.), dotted line-pH. Right column depicts the CEX-HPLC analyses of the individual peaks pooled from the respective preparative chromatographic runs on the left. A-H in CEX-HPLC analyses depict different monomeric charge variants.

By comparing the three different gradient types in FIG. 5, linear salt gradient elution only depicts one eluted peak whereas the other two show a main peak and a shoulder. This indicates that salt gradient is the least efficient system among the three methods tested here. For the pH gradient and hybrid gradient elution, removal of certain charge variants can be attained in both set-ups but the latter depicts better resolved shoulder which contains basic charge variants. Also from the CEX-HPLC analyses, it is seen that the shoulder peak 3 in the hybrid gradient contains two basic charge variants (G and H) compared to the pH gradient (F, G, and H), which indicates a better separation of the isoproteins using the hybrid gradient compared to conventional pH gradient elution system.

The following data compares the capacity as well as the corresponding charge variants separation efficiencies of the linear pH gradient and opposite pH-salt hybrid gradient elution.

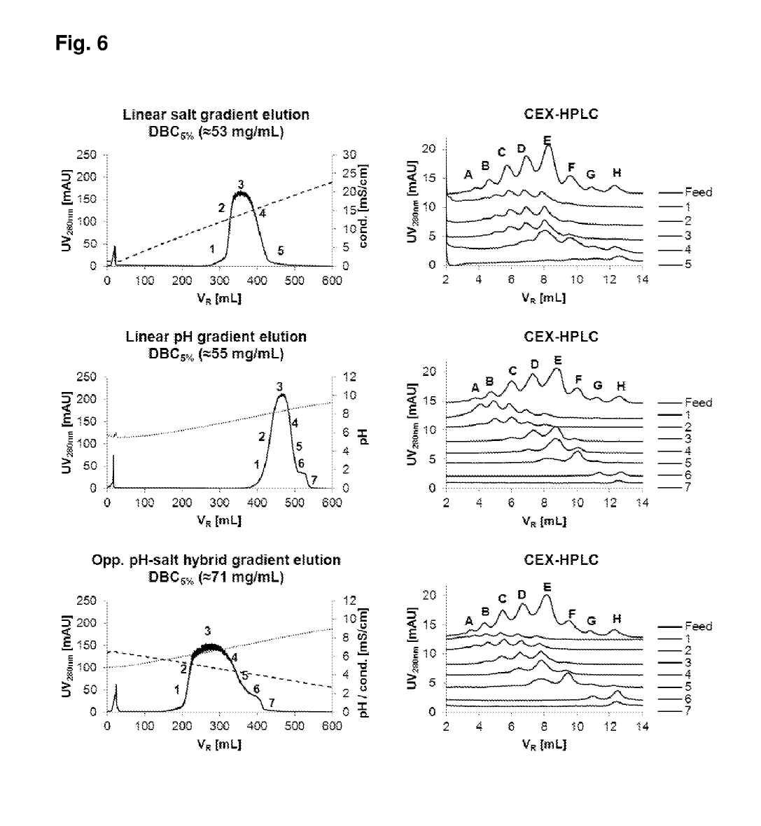

FIG. 6: (FIG. 6) Left column depicts the respective preparative chromatographic runs of linear salt gradient elution 0-0.25 M NaCl, pH 5, linear pH gradient elution pH 5-9.5, 0 M NaCl, and opposite pH-salt hybrid gradient pH 5-9.5, 0.05-0 M NaCl on Eshmuno® CPX, using 5% breakthrough (DBC_{5%}). Gradient slope—690 CV. Dashed line-conductivity (cond.), dotted line-pH. Right column depicts the CEX-HPLC analyses of the individual peaks pooled from the respective preparative chromatographic runs on the left. A-H in CEX-HPLC analyses depict different monomeric charge variants. Protein recovery for every run is >90%.

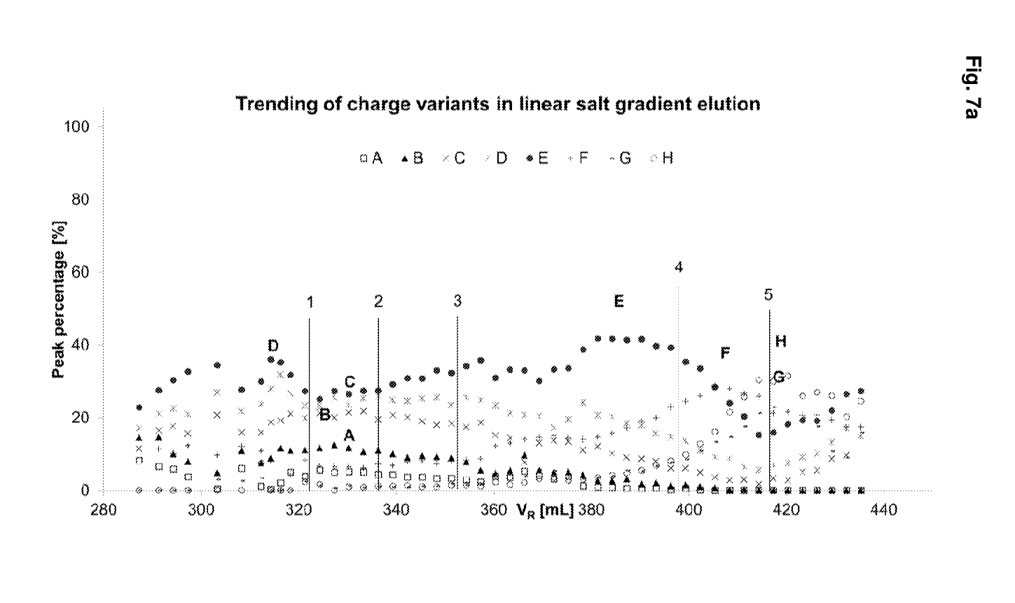

FIGS. 7a-7c: (FIGS. 7a-7c) Summed percentages of the individual charge variants in the eluted peaks of the respective gradient types shown in FIG. 6. A-H show the maxima of the individual charge variants shown in CEX-HPLC of FIG. 6 along the gradient. Straight lines labeled with numbers (1-7) show the positions where the fraction pools in FIG. 6 are taken.

Compared to the DBC of classical linear salt and linear pH gradient elution (DBC_{5%}≈53-55 mg/mL packed resins), the DBC of mAb B is significantly higher (DBC_{5%}≈71 mg/mL packed resins) when opposite pH-salt hybrid gradient with increasing pH and descending salt gradient is used (see FIG. 6). According to the changes of charge variants along the elution gradient (see FIG. 7), it is observed that in the linear salt gradient, acidic charge variants (A, B, C, D) and basic charge variants (G, H) are lumped up at the starting of the gradient and at the end of the gradient, respectively, thus leading to an inefficient separation of the charge variants. On the contrary, these charges variants are distributed evenly along the pH gradient and hybrid gradient, respectively. It should be noted that the slightly better distribution of the charge variants along the pH gradient compared to the hybrid gradient was because less proteins could be loaded onto the column using the pH gradient buffer before DBC_{5%} was reached. As shown in Example 1 (see FIGS. 3a-3d (C) and (E)), if similar amount of proteins as that used in the hybrid gradient (i.e. ˜71 mg/mL per packed resins) are loaded onto the column using the pH gradient buffers at elevated salt concentration, the separation of charge variants will be worse than the hybrid gradient. Hence, it is reasonable to conclude that the hybrid gradient improves DBC of the proteins without a loss in isoproteins separation efficiency compared to classical pH gradient method.

The experiments show, that the charge variants separation can be improved if a mixture containing less of such species is used. Thus, the shoulder peak 5-7 of the opposite pH-salt hybrid gradient in FIG. 6 is pooled and combined to form a feed with less charge variants (E, F, G, and H) and is re-chromatographed using similar experimental set-ups.

The following data show the results of the re-chromatographed feed containing E, F, G and H charge variants.

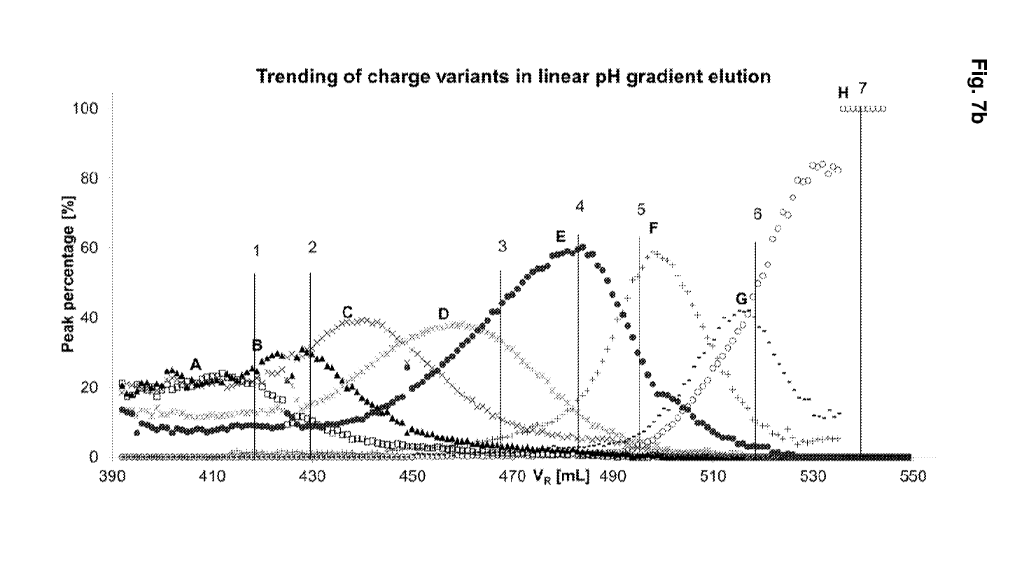

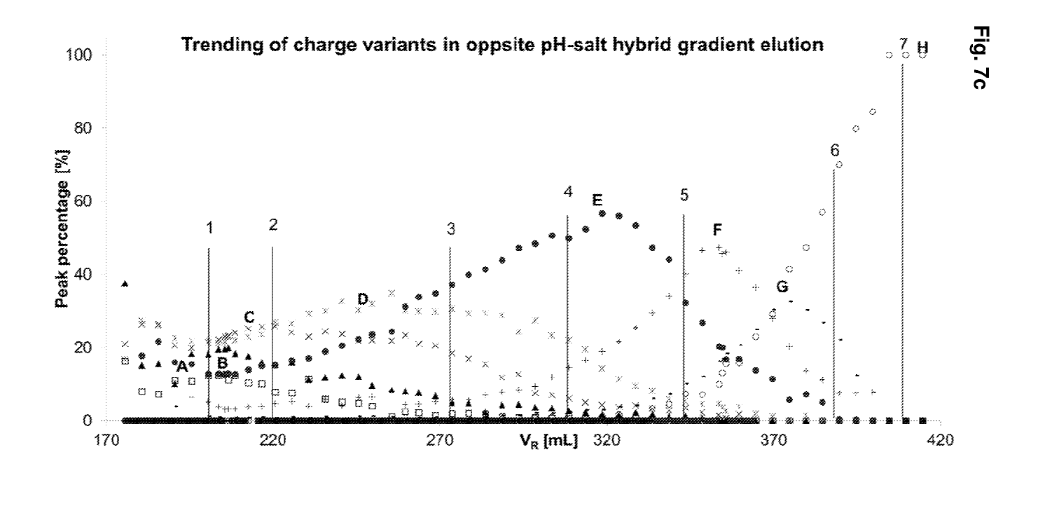

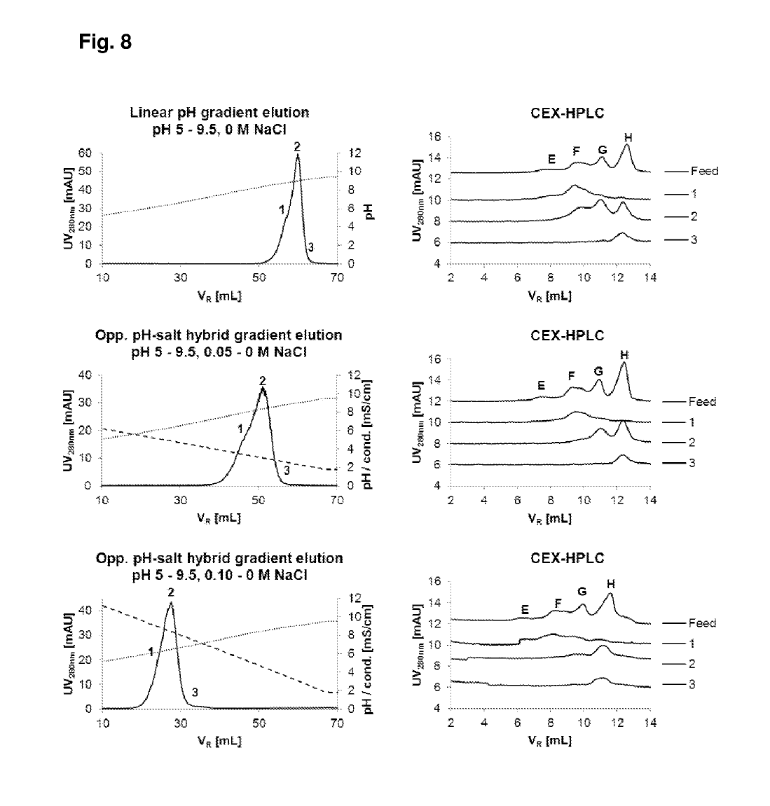

FIG. 8: (FIG. 8) Re-chromatography of the feed containing the charge variants E, F, G, and H pooled from the shoulder peak 5-7 of the opposite pH-salt hybrid gradient in FIG. 6. Left column depicts the respective preparative chromatographic runs of linear pH gradient elution pH 5-9.5, 0 M NaCl and opposite pH-salt hybrid gradient pH 5-9.5, 0.05-0 M NaCl/0.10-0 M NaCl (from top to bottom) on Eshmuno® CPX. Dashed line-conductivity (cond.), dotted line-pH. Right column depicts the CEX-HPLC analyses of the individual peaks pooled from the respective preparative chromatographic runs on the left. E-H in CEX-HPLC analyses depict different monomeric charge variants.

Best resolution between shoulder peak 1 and the main peak 2 is achieved when opposite pH-salt hybrid gradient with 0.05 M NaCl is used (middle row in FIG. 8). Nevertheless, CEX-HPLC results show that the main peak 2 in the hybrid gradient with 0.10 M NaCl contains only one main charge variant H, indicating that this system has the most effective charge variants separation. Amongst the three systems, hybrid gradient system outperforms linear pH gradient system in terms of resolution and charge variants removal efficiency.

### Example 3

Preparative Separation of mAb B Fc, Fab, ⅔ Fragment, and Monomeric Species Using IEC

The preparative chromatographic runs were performed as follows:

Equipment: ÄKTApurifier 100

Column: Eshmuno® CPX, Merck Millipore, mean particle size 50 μm, ionic capacity 60 μmol/mL, column dimension 8 i.d.×20 mm (1 mL)

Feed: MAb B native monomer spike with Fc/Fab, and ⅔ fragment

Mobile phase:
(A) For linear pH gradient, buffer A consisted of 12 mM acetic acid, 10 mM MES, and 10 mM MOPS whilst buffer B consisted of 6 mM MOPS, 6 mM HEPES, 10 mM TAPS, and 9 mM CHES. Buffer A and B were adjusted to pH 5 and 9.5, respectively with NaOH.
(B) For opposite pH-salt hybrid gradient with ascending pH and descending salt gradient, same buffer components as (A) are used but certain amount of sodium chloride (50 mM or 100 mM) is added into buffer A while none is added to buffer B. Both buffers are adjusted to pH 5 and 9.5, respectively with NaOH.

Gradient Slope: 60 CV (1 mL/CV)

Flow rate: 1 mL/min (=119 cm/h)

Protein load: 1 mg/mL, otherwise will be stated in the descriptions of the figures

CIP: 0.5 M NaOH (3-5 CV)

Step Elution:

Flow rate: 1 mL/min (=119 cm/h) was used to bind protein; 3 mL/min (=358 cm/h) is used to elute protein

Protein load: 30 mg/mL

Cleaning-In-Place (CIP): 0.5 M NaOH (3-5 CV)

Buffer A and B as stated in (B) (see mobile phase) are used. Zero % buffer

B is used for protein binding. For protein elution different steps are generated by mixing buffer A and B at different concentrations as follows:

| | Buffer |
|---|---|
| Step | B [%] |
| 1 | 28.5 |
| 2 | 34 |
| 3 | 46 |
| 4 | 63 |
| 5 | 76 |

Analytics were performed as follows:

Equipment: ÄKTAmicro

SE-HPLC was performed using Superdex™ 200 Increase 10/300 GL, GE Healthcare, column dimension 10 i.d.×300 mm, mean particle size 8.6 μm. Buffer used consist of 50 mM NaH₂PO₄ and 300 mM NaCl, pH 7. Isocratic elution at a flow rate of 0.5 mL/min is used. Injection volume varies from 40 μL to 100 μL.

Results:

The following data show that the process of the present invention has a particular advantage over a process using a pH gradient for the separation of native mAb from other soluble size variants like ⅔ fragments, Fc and Fab using CEX.

FIG. 9: (FIG. 9) Left column depicts the respective preparative chromatographic runs of linear pH gradient elution pH 5-9.5, 0 M NaCl and opposite pH-salt hybrid gradient pH 5-9.5, 0.05-0 M NaCl on Eshmuno® CPX. Dashed line—conductivity (cond.), dotted line-pH. Right column depicts the SE-HPLC analyses of the individual peaks pooled from the respective preparative chromatographic runs on the left. MAb—native monomeric mAb B, ⅔ Fg.-⅔ fragment, Fc—crystallizable fragment, Fab-antigen-binding fragment.

Although the separation results are convincing it needs an trained expert when interpreting the Fc and Fab peaks in the SE-HPLC results in FIG. 9. Fc (V_{R}≈15 mL) appears as a shoulder before Fab (V_{R}≈15.5 mL). For the SE-HPLC analysis of the chromatographic run using linear pH gradient elution, fraction pool 1 and 2 contain only Fc whereas Fab is found in fraction pool 4 and 5. Likewise, for the chromatographic run using opposite pH-salt hybrid gradient elution, the corresponding SE-HPLC results show that fraction pool 1 contains mainly Fab whereas fraction pool 2 is a mixture of both Fc and Fab.

By comparing both chromatographic runs on the left in FIG. 9, despite the higher number of resolved peaks obtained using linear pH gradient elution, the product peak (i.e. peak 6 in the chromatogram on the top left) overlaps with the Fab peak (i.e. peak 5 in the same chromatogram). On the contrary, although less peaks are resolved in the opposite pH-salt hybrid gradient elution, the product peak (i.e. peak 4 in the chromatogram on the bottom left) can be cut off very well from the other impurities peaks which provide a wider window for the elution of the product using a step elution. Here, it is also clear that by employing a descending salt gradient in the ascending pH gradient, the interaction between Fab and the stationary phase is strongly suppressed thereby leading to a complete exclusion of this peak from the product peak. In the pH gradient elution (top left in FIG. 9), the Fab species is eluted after Fc and ⅔ fragment. However, in the hybrid gradient elution (bottom left in FIG. 9), the Fab species is eluted prior to Fc and ⅔ fragment.

Since the native monomeric mAb used in this study is the same as that used in Example 2, peak 4 and 5 of the opposite pH-salt hybrid gradient elution (bottom left in FIG. 9) resemble the eluted peaks in FIG. 5 (bottom left) and previously it has been shown that charge variants are separated in FIG. 5. Therefore, by combining both results of example 2 and 3, it is proven that the opposite pH-salt hybrid gradient can be used to separate both charge and size variants simultaneously, which again confirms the result shown in example 1.

The following data compare corresponding charge variants separation efficiencies of the linear pH gradient and opposite pH-salt hybrid gradient elution at higher loading.

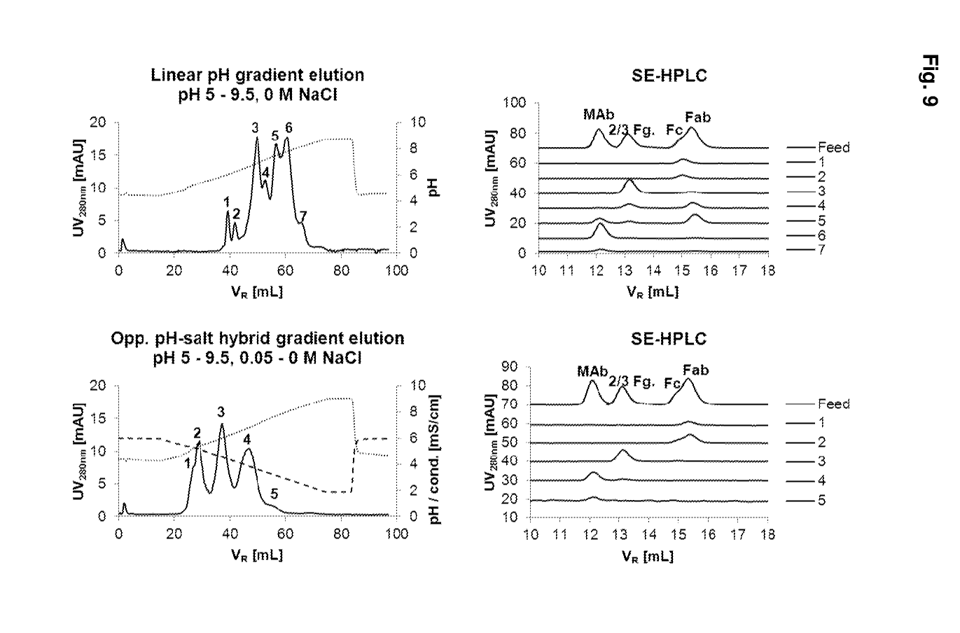

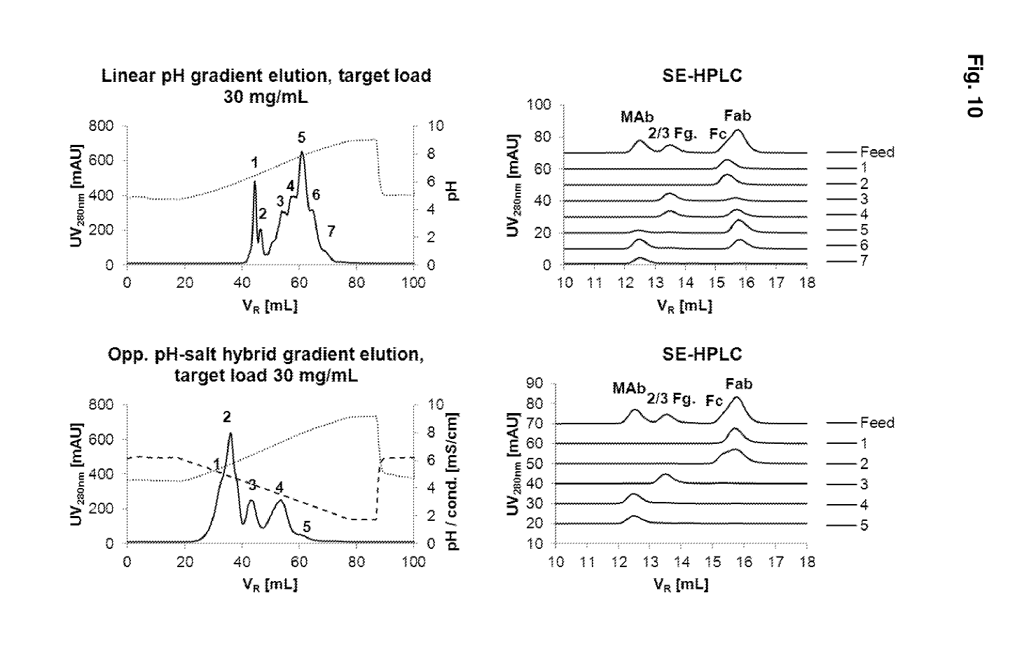

FIG. 10: (FIG. 10) Left column depicts the respective preparative chromatographic runs of linear pH gradient elution pH 5-9.5, 0 M NaCl and opposite pH-salt hybrid gradient pH 5-9.5, 0.05-0 M NaCl on Eshmuno® CPX, using a load of 30 mg/mL packed resins. Dashed line-conductivity (cond.), dotted line-pH. Right column depicts the SE-HPLC analyses of the individual peaks pooled from the respective preparative chromatographic runs on the left. MAb—native monomeric mAb B, ⅔ Fg.-⅔ fragment, Fc—crystallizable fragment, Fab-antigen-binding fragment.

In FIG. 10, multiproduct separation efficiency is tested at high loading (=30 mg/mL packed resins). The same separation results as shown in FIG. 9 are reproduced. It is noted here, that the feed used in this experiment contained slightly higher percentages of Fc and Fab compared to the feed used in FIG. 9. Nevertheless, the elution profiles and the eluent sequences are identical in both cases; with pH gradient elution showing a higher number of resolved peaks but less efficiently separated product pool (peak 6 of top left chromatogram in FIG. 10) whereas it is the opposite for hybrid gradient elution (peak 4 of bottom left chromatogram in FIG. 10). Again, it is shown that the hybrid gradient elution system can be used at high protein loading for purification.

The following shows the transfer of separation process from hybrid pH-salt gradient elution into a series of stepwise elution by using the same buffer systems.

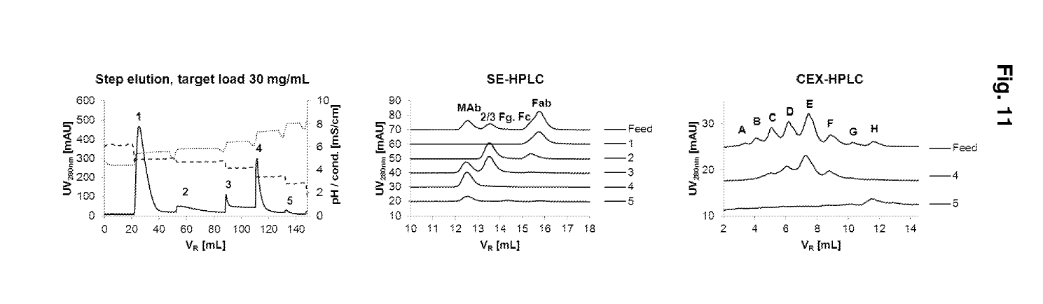

FIG. 11: (FIG. 11) Left column depicts the multiproduct separation using step elution on Eshmuno® CPX. Peak 1 is eluted in first step (28.5% buffer B), peak 2 in the second step (34% buffer B), peak 3 in the third step (46% buffer B), peak 4 in the fourth step (63% buffer B), and peak 5 in the fifth step (76%). Dashed line—conductivity (cond.), dotted line—pH. Middle and right columns are the HPLC analyses of the individual peaks pooled from the preparative chromatographic run on the left. MAb—native monomeric mAb B, ⅔ Fg.-⅔ fragment, Fc—crystallizable fragment, Fab-antigen-binding fragment. A-H in CEX-HPLC analyses depict different monomeric charge variants.

Similar to Example 1, the separation process is transferred from hybrid gradient elution system into a series of stepwise elution. According to the SE-HPLC results in FIG. 11, peak 1 contains Fab with a purity of >99% and a yield of ˜91% whereas peak 4 contains mAb with a purity of >99% and a yield of ˜70%. Peak 2 comprised of ˜75% purity of ⅔ fragments together with ˜25% purity of Fc. About 50% yield of ⅔ fragments is eluted in peak 2, whereas the other half is found in peak 3, together with some mAbs. Also in peak 4 and 5, charge variants separation is observed, depicted in the CEX-HPLC results in FIG. 10 where the acidic variants A, B, C, D, E, and F are found in fraction pool 4 and basic variants G and H are found in the final fraction pool 5. The separation of charge variants using step elution reconfirms the observation in hybrid gradient elution shown in Example 2 that the corresponding buffer system is suitable for the separation of acidic from basic charge variants.

In summary, Example 3 shows a universal suitability of the present opposite hybrid pH-salt gradient system for size variants and charge variants separation, which works at high loading and which is also easily transferable into a series of stepwise elution.

### Example 4

Preparative Separation of mAb B Fc, Fab, ⅔ Fragment, and Monomeric Species Using MMC

The preparative chromatographic runs are performed as follows:

Equipment: ÄKTApurifier 100

Column: Capto® MMC, GE Healthcare, mean particle size 75 μm, ionic capacity 70-90 μmol/mL, column dimension 8 i.d.×20 mm (1 mL)

Feed: MAb B native monomer spike with Fc/Fab, and ⅔ fragment

Mobile phase:
(A) For linear pH gradient, buffer A consists of 12 mM acetic acid, 10 mM MES, and 10 mM MOPS whilst buffer B consists of 6 mM MOPS, 6 mM HEPES, 10 mM TAPS, and 9 mM CHES. Buffer A and B are adjusted to pH 5 and 9.5, respectively with NaOH.
(B) For opposite pH-salt hybrid gradient with ascending pH and descending salt gradient, same buffer components as (A) are used but a certain amount of sodium chloride (50 mM or 100 mM) is added into buffer A while none is added to buffer B. Both buffers are adjusted in a pH range between pH 5 and 9.5, respectively with NaOH.

Gradient Slope: 60 CV (1 mL/CV)

Flow rate: 1 mL/min (=119 cm/h)

Protein load: 1 mg/mL

CIP: 0.5 M NaOH (3-5 CV)

Analytics are performed as follows:

Equipment: ÄKTAmicro

SE-HPLC is performed using Superdex™ 200 Increase 10/300 GL, GE Healthcare, column dimension 10 i.d.×300 mm, mean particle size 8.6 μm. Buffer used consists of 50 mM NaH₂PO₄ and 300 mM NaCl, pH 7. Isocratic elution at a flow rate of 0.5 mL/min is used. Injection volume varied from 40 μL to 100 μL.

Results:

The following data are collected showing the advantage of the present invention over pH gradient for the separation of native mAb from other soluble size variants like ⅔ fragments, Fc and Fab using MMC.

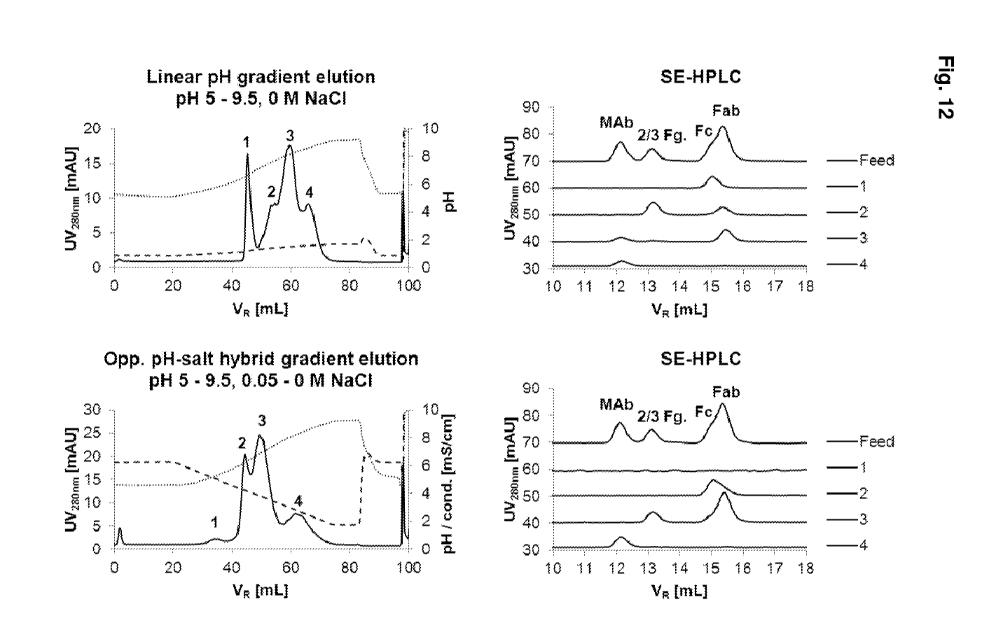

FIG. 12: (FIG. 12) Left column depicts the respective preparative chromatographic runs of linear pH gradient elution pH 5-9.5, 0 M NaCl and opposite pH-salt hybrid gradient pH 5-9.5, 0.05-0 M NaCl on Capto® MMC. Dashed line—conductivity (cond.), dotted line-pH. Right column depicts the SE-HPLC analyses of the individual peaks pooled from the respective preparative chromatographic runs on the left. MAb—native monomeric mAb B, ⅔ Fg.-⅔ fragment, Fc—crystallizable fragment, Fab-antigen-binding fragment.

According to FIG. 12, linear pH gradient results in 4 peaks (peak 1-4) in which proteins were detected in the SE-HPLC whereas opposite pH-salt hybrid gradient resulted in 3 peaks (peak 2-4) with proteins. Nevertheless, the product peak (peak 4) is better resolved from the other peaks (i.e. the impurities) using the opposite pH-salt hybrid gradient compared to the linear pH gradient. This is consistent with results from separation of isoproteins on CEX (see FIG. 9), which also means that the window of optimization to develop a step elution for product separation from the impurities is wider using the opposite pH-salt hybrid gradient system compared to the classical linear pH gradient approach.

Therefore, it is shown that the present invention is suitable for the separation of isoproteins not only in IEC, but also in MMC.

1. Method for separating and purifying a protein from a mixture of proteins, by the steps:
a) providing a sample comprising at least two different proteins,
b) applying this mixture to an ion exchange material with a total protein load ≥5 mg/ml, especially ≥30 mg/ml, in particular ≥60 mg/ml,
c) running an opposite pH-salt gradient by an ascending pH and descending salt concentration to separate proteins, or vice versa
running a descending pH and an ascending salt concentration, or running a increasing pH gradient, or running a decreasing pH gradient,
and
d1) using the separation data from c) to define and run a step elution profile for protein separation
and
e1) separating the proteins in a stepwise elution;
or
d2) separating the proteins in a gradient elution.

2. Method for separating and purifying a protein from a mixture of proteins according to claim 1, by the steps:
a) providing a sample comprising at least two different proteins,
b) applying this mixture to an ion exchange material with a total protein load ≥5 mg/ml, especially ≥30 mg/ml, in particular ≥60 mg/ml,
c) running an opposite pH-salt gradient by an ascending pH and descending salt concentration to separate proteins, or vice versa running a descending pH and an ascending salt concentration, or running a increasing pH gradient, or running a decreasing pH gradient,
d2) separating the proteins in a gradient elution.

3. Method according to claim 1, wherein a mixture of proteins is adsorbed or bound to and eluted from a ion exchange material.

4. Method according to 1, wherein a mixture of proteins is adsorbed and eluted from a cation exchange material.

5. Method according to 1, wherein a mixture of proteins is adsorbed and eluted from a anion exchange material.

6. Method according to claim 1, which comprises steps d1) and e1), wherein a mixture of proteins is adsorbed or bound and eluted from a mixed mode chromatography material.

7. Method according to claim 1, wherein in c) opposite pH-salt gradient is induced by a buffering system using MES, MOPS, CHAPS and comparable biological buffers and a conductivity alteration system using sodium chloride.

8. Method according to claim 1 wherein in c) the pH is changed in the range from 4-10.5 and the salt concentration in the range of 0-1M salt.

9. Method according to claim 1, wherein a pH gradient is induced by applying a buffer system adjusted to pH 5 and 9.5.

10. Method according to claim 1 wherein a salt gradient is induced in a concentration range between 0-0.25 M.

11. Method according to claim 1, wherein a pH gradient is induced by applying a buffer system of at least two buffer solutions,
whereby adsorption or binding of proteins takes place in presence of one buffer solution and elution takes place in presence of increasing concentrations of the other buffer solution, while the pH value is ascending and the salt concentration is descending simultaneously.

12. Method according to claim 1, wherein a pH gradient is induced by applying a buffer system of at least two buffer solutions,
whereby adsorption or binding of proteins takes place in presence of one buffer solution and elution takes place in presence of increasing concentrations of the other buffer solution while pH is descending and the salt concentration is ascending simultaneously.

13. Method according to claim 1, wherein proteins, particularly monoclonal antibodies (mAB), are separated and purified from its associated charge variants, glycosylation variants, and/or soluble size variants, dimers and aggregates, monomers, ⅔ fragments, ¾ fragments, fragments in general, antigen binding fragments (Fab) and Fc fragments.

14. Method for separating and purifying a protein from a mixture of proteins according to claim 1, by the steps:
a) providing a sample comprising at least two different proteins,
b) applying this mixture to an ion exchange material with a total protein load ≥5 mg/ml, especially ≥30 mg/ml, in particular ≥60 mg/ml,
c) running an opposite pH-salt gradient by an ascending pH and descending salt concentration to separate proteins, or vice versa
running a descending pH and an ascending salt concentration, or running a increasing pH gradient, or running a decreasing pH gradient,
d1) using the separation data from c) to define and run a step elution profile for protein separation
and
e1) separating the proteins in a stepwise elution.
